(19) United States
(12) Patent Application Publication
(10) Pub. No.: US 2007/0135472 A1
(43) Pub. Date: Jun. 14, 2007

(54) Novel crystalline forms of desloratadine and processes for their preparation

(75) Inventors: Bobba Venkata Siva Kumar, Koper Khairne (IN); Sanjay Anantha Kale, Navi (IN); Raju Baban Choudhari, Kalyan (west) (IN); Nitin Sharad Chandra Pradhan, Thane (W) (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/607,229

(22) Filed: Dec. 1, 2006

Related U.S. Application Data
(60) Provisional application No. 60/756,275, filed on Jan. 4, 2006.

(30) Foreign Application Priority Data
Dec. 1, 2005 (IN) 1487/MUM/2005

(51) Int. Cl. A61K 31/473

(52) U.S. Cl. 514/290; 546/93

(57) ABSTRACT

Novel polymorph Forms III and V of desloratadine are provided. Pharmaceutical compositions containing such polymorphs are also provided.

### PRIORITY

This application claims the benefit under 35 U.S.C. §119 to U.S. Provisional Application No. 60/756,275, filed on Jan. 4, 2006, and entitled “CRYSTALLINE FORMS OF DESLORATADINE AND PROCESSES FOR THEIR PREPARATION”, and to Indian Provisional Application No. 1487/MUM/2005, filed on Dec. 1, 2005, and entitled “NOVEL CRYSTALLINE FORMS OF DESLORATADINE AND PROCESSES FOR THEIR PREPARATION”, the contents of each of which are incorporated by reference herein.

### BACKGROUND OF THE INVENTION

1. Technical Field

The present invention generally relates to crystalline forms III and V of desloratadine and processes for their preparation.

2. Description of the Related Art

Desloratadine, also known as 8-chloro-6,11-dihydro-11-(4-piperidylidene)-5H-benzo[5,6]cyclohepta[1,2-b]pyridine, is represented by the structure of Formula I: 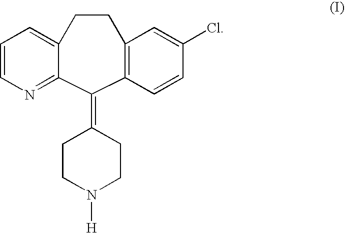
Desloratadine is an active metabolite of loratadine, which is orally active’ long-acting histamine H₁ receptor antagonist. It belongs to a second generation of H₁ histamine receptor antagonists that are thought to offer advantages over the first generation compounds. Desloratadine is a long-acting tricyclic histamine antagonist with selective H₁-receptor histamine antagonist activity. Desloratadine is currently marketed under the trade name Clarinex® in the United States. Clarinex is indicated for the relief of the nasal and non-nasal symptoms of seasonal allergic rhinitis, nasal and non-nasal symptoms of perennial allergic rhinitis and symptomatic relief of pruritus, reduction in the number and size of hives in patients with chronic idiopathic urticaria. See, e.g., The Merck Index, Thirteenth Edition, 2001, pp. 514, monograph 2939; and Physician's Desk Reference, “Clarinex,” 60th Edition, pp. 3009-12 (2005).

U.S. Pat. No. 4,659,716 (“the '716 patent”) discloses desloratadine. The '716 patent further discloses methods for preparing and administering desloratadine and its pharmaceutically acceptable salts. U.S. Pat. No. 4,282,233 also discloses loratadine.

U.S. Pat. No. 6,506,767 (“the '767 patent”) discloses crystalline polymorph forms I and II of desloratadine and their use in pharmaceutical compositions. The '767 patent further discloses that polymorph form 1 can be essentially free of polymorph form 2 and is defined as containing less than about 1% of form 2 as measured by infrared spectral analysis on a FTIR spectrometer and that polymorph form 2 can be substantially free of polymorph form 1 and is defined as containing less than about 15% of form I.

PCT publication WO 2004/029039 (“the '039 application”), herein incorporated by reference, discloses a process for preparing desloratadine including the decarboethoxylation of loratadine.

Polymorphic forms occur where the same composition of matter crystallizes in a different lattice arrangement resulting in, for example, different thermodynamic properties and stabilities specific to the particular polymorph form. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. It is well known that the crystalline polymorph form of a particular drug is often an important determinant of the drug's ease of preparation, stability, solubility, storage stability, ease of formulation and in vivo pharmacology. Thus, in cases where two or more polymorph substances can be produced, it may be desirable to have a method to make both polymorphs in pure form. In deciding which polymorph is preferable, the numerous properties of the polymorphs must be compared and the preferred polymorph chosen based on the many physical property variables. It is entirely possible that one polymorph form can be preferable in some circumstances where certain aspects such as ease of preparation, stability, etc are deemed to be critical. In other situations, a different polymorph maybe preferred for greater solubility and/or superior pharmacokinetics. Polymorphic forms of a compound can be distinguished in a laboratory by X-ray diffraction (“XRD”) spectroscopy and by other methods such as infrared (“IR”) spectrometry. Additionally, polymorphic forms of the same drug substance or active pharmaceutical ingredient, can be administered by itself or formulated as a drug product (also known as the final or finished dosage form), and are known in the pharmaceutical art to affect, for example, the solubility, stability, flowability, tractability and compressibility of drug substances and the safety and efficacy of drug products. New polymorphic forms of desloratadine have now been discovered.

### SUMMARY OF THE INVENTION

In accordance with one embodiment of the present invention, desloratadine in polymorph Form III is provided.

In accordance with a second embodiment of the present invention, desloratadine in polymorph Form III and having an X-ray powder diffraction (XRD) pattern substantially in accordance with FIG. 1 is provided.

In accordance with a third embodiment of the present invention, desloratadine in polymorph Form III and exhibiting a characteristic peak (expressed in degrees 2θ±0.2°θ) at about 18.40 is provided.

In accordance with a fourth embodiment of the present invention, desloratadine in polymorph Form III and exhibiting characteristic peaks (expressed in degrees 2θ±0.2°θ) at approximately one or more of the positions: about 10.67, about 12.45, about 17.08, about 17.97, about 18.40, about 18.79, about 19.69 and about 21.49 is provided.

In accordance with a fifth embodiment of the present invention, a pharmaceutical composition is provided comprising a therapeutically effective amount of desloratadine in polymorph Form III.

In accordance with a sixth embodiment of the present invention, desloratadine in polymorph Form V is provided.

In accordance with a seventh embodiment of the present invention, desloratadine in polymorph Form V and having an XRD pattern substantially in accordance with FIG. 2 is provided.

In accordance with an eighth embodiment of the present invention, desloratadine in polymorph Form V and exhibiting characteristic peaks (expressed in degrees 2θ±0.2°θ) at approximately one or more of the positions: about 15.07 and about 23.84 is provided.

In accordance with a ninth embodiment of the present invention, desloratadine in polymorph Form V and exhibiting characteristic peaks (expressed in degrees 2θ±0.2°θ) at approximately one or more of the positions: about 8.46, about 11.80, about 15.07, about 16.95, about 18.00, about 19.75, about 20.66, about 21.86 and about 23.84 is provided.

In accordance with a tenth embodiment of the present invention, a pharmaceutical composition is provided comprising a therapeutically effective amount of desloratadine in polymorph Form V.

Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “pharmaceutically acceptable” means that which is useful in preparing a pharmaceutical composition that is generally non-toxic and is not biologically undesirable and includes, but is not limited to, that which is customarily utilized for veterinary use and/or human pharmaceutical use.

The term “composition” includes, but is not limited to, a powder, a solution, a suspension, a gel, an ointment, an emulsion and/or mixtures thereof. The term “composition” is intended to encompass a product containing the specified ingredient(s) in the specified amount(s), as well as any product, which results, directly or indirectly, from combination of the specified ingredients in the specified amounts. A “composition” may contain a single compound or a mixture of compounds. A “compound” is a chemical substance that includes molecules of the same chemical structure.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the crystalline solids described herein, additional active ingredient(s), and pharmaceutically acceptable excipients.

When referring to a chemical reaction, the terms “treating”, “contacting” and “reacting” are used interchangeably herein and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “substantially free of” in reference to a composition, as used herein, means that the substance from which the composition is free of cannot be detected by methods known to those skilled in the art.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in tablet granulations. Such compounds include, by way of example and without limitation, acacia alginic acid, tragacanth, carboxymethylcellulose sodium, poly(vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

The term “diluent” or “filler” as used herein is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, polyvinylpyrrolidone (PVP), tyloxapol (a nonionic liquid polymer of the alkyl aryl polyether alcohol type, also known as superinone or triton), combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### BRIEF DESCRIPTION OF THE DRAWINGS

FIG. 1 is a characteristic powder XRD pattern of polymorph Form III of desloratadine.

FIG. 2 is a characteristic powder XRD pattern of polymorph Form V of desloratadine.

### DETAILED DESCRIPTION OF THE INVENTION

The present invention is directed to novel polymorphs of desloratadine, designated polymorph Forms III and V. The desloratadine used in the present invention may be obtained from loratadine, by hydrolysis of the carbamate, preferably under basic conditions. Loratadine itself may be prepared from N-methyl desloratadine by removing N-methyl group of N-methyl desloratadine by formation of the carbamate through reaction with a haloformate. The haloformate used may be an alkyl or aryl formate, with optional halogen substituted at first and/or second position of the formate, i.e., 2-chloroethyl-chloroformate. The carbamate may be prepared in an anhydrous C₅-C₁₂ hydrocarbon, such as toluene. When N-methyl desloratadine is used as a stating material, loratadine may or may not be isolated in preparation of desloratadine. The removal of the carbamate group of loratadine may be carried out with a base at elevated temperature. A preferred temperature is reflux temperature. The inorganic base may be an alkali metal hydroxide, such as lithium hydroxide, magnesium hydroxide, sodium hydroxide, potassium hydroxide, preferably sodium hydroxide. The amount of inorganic base may vary from about 0.5 to about 2.0 w/w equivalents with respect to the starting material loratadine, preferably from about 1 to about 1.5 w/w equivalents, and more preferably about 1:1.06 w/w equivalents. A preferred solvent is a C₁-C₄ alcohol such as 2-propanol. If, desired, the desloratadine used herein may be in a polymorphic form, e.g., Form I and/or II, or mixtures thereof which can be prepared by techniques known in the art.

Crystallinity of the polymorphs may be measured using methods familiar to those skilled in the art. The novel polymorphs of the present invention were characterized by X-ray powder diffraction. The X-Ray powder diffraction spectrums for polymorph Forms III and V of desloratadine were measured by an X-ray powder Diffractometer equipped with a Cu-anode (λ=1.54 Angstrom), X-ray source operated at 45 kV, 40 mA and a nickel filter is used to strip K-beta radiation. Two-theta calibration is performed using an NIST SRM 640c Si standard. The sample was analyzed using the following instrument parameters: measuring range=2-50° 2θ; step width=0.017°; and measuring time per step=5 sec.

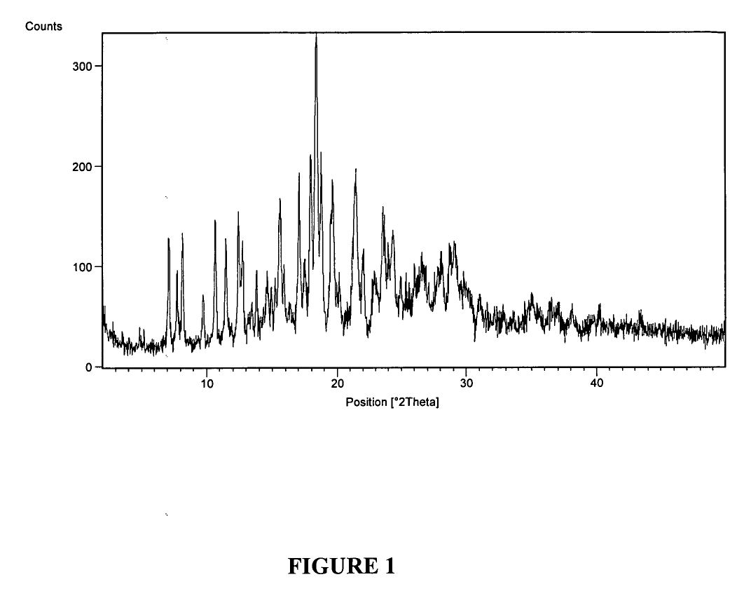

One embodiment of the present invention is directed to a novel polymorph of desloratadine substantially in crystalline Form III. The crystalline Form III of desloratadine has at least an XRD pattern substantially in accordance with FIG. 1. In one embodiment, desloratadine in polymorph Form III exhibits a characteristic peak (expressed in degrees 2θ±0.2°θ) at about 18.40. In another embodiment, desloratadine in polymorph Form III exhibits characteristic peaks (expressed in degrees 2θ±0.2°θ) at approximately one or more of the positions: about 10.67, about 12.45, about 17.08, about 17.97, about 18.40, about 18.79, about 19.69 and about 21.49. Generally, desloratadine in polymorph Form III exhibits an XRD pattern having the approximate values set forth in tabular form in Table 1 below:

TABLE 1

| | d-Value | Relative |
|---|---|---|
| 2θ | Angstrom | Intensity (%) |
| 7.10 | 12.45 | 37.33 |
| 7.74 | 11.42 | 24.51 |
| 8.20 | 10.79 | 34.23 |
| 9.73 | 9.09 | 16.36 |
| 10.67 | 8.29 | 41.35 |
| 11.47 | 7.71 | 28.55 |
| 12.45 | 7.11 | 42.18 |
| 12.76 | 6.94 | 30.62 |
| 13.80 | 6.42 | 21.55 |
| 14.60 | 6.07 | 13.92 |
| 15.56 | 5.70 | 38.90 |
| 17.08 | 5.19 | 47.81 |
| 17.49 | 5.07 | 22.49 |
| 17.97 | 4.94 | 57.26 |
| 18.40 | 4.82 | 100.00 |
| 18.79 | 4.72 | 49.06 |
| 19.69 | 4.51 | 46.73 |
| 20.19 | 4.40 | 14.05 |
| 21.49 | 4.13 | 48.96 |
| 22.01 | 4.04 | 21.54 |
| 22.84 | 3.89 | 13.41 |
| 23.55 | 3.78 | 34.91 |
| 24.36 | 3.65 | 28.81 |
| 26.66 | 3.34 | 17.08 |
| 28.13 | 3.17 | 18.68 |
| 28.72 | 3.11 | 24.11 |
| 30.98 | 2.89 | 9.18 |
| 34.96 | 2.57 | 9.46 |

Generally, crystalline Form III of desloratadine can be obtained by at least:

(a) reacting loratadine with an inorganic base in an organic solvent to obtain a reaction mixture comprising desloratadine;

(b) heating the reaction mixture;

(c) extracting desloratadine in a water immiscible solvent;

(d) stirring the product of step (c) for about 80 to about 100 hours; and

(e) recovering desloratadine in polymorph Form III.

The inorganic base may be selected, but not limited to, alkali metal hydroxides such as lithium hydroxide, magnesium hydroxide, sodium hydroxide, potassium hydroxide, and the like. Useful organic solvents include, but are not limited to, alcohols, ketones, aromatic and aliphatic hydrocarbons, esters, ethers, nitrites, acids, water or mixtures thereof. The dissolution can be carried out at a temperature ranging from about 0° C. to about 150° C. and preferably at room temperature.

Useful water immiscible solvents include, but are not limited to, straight or branched chain aliphatic hydrocarbons, chlorinated solvents, cyclic solvents having 6 to 15 carbon atoms and the like and mixtures thereof. Exemplary water-immiscible solvents include, but are not limited to, dichloromethane, cyclohexane, n-heptane, hexane, octanol, n-decane, decalene and mixtures thereof. Isolation of the product can be achieved by techniques know in the art. For example, isolation may be accomplished by concentration by distillation, distillation under vacuum, or distillation in Rota evaporator, cooling, addition of a second solvent, filtration, filtration under vacuum, decantation and centrifugation or a combination thereof.

The product obtained may be further dried to achieve the desired residual solvent level. For example, the product may be further dried in a tray drier, or dried under vacuum and/or in a Fluid Bed Drier.

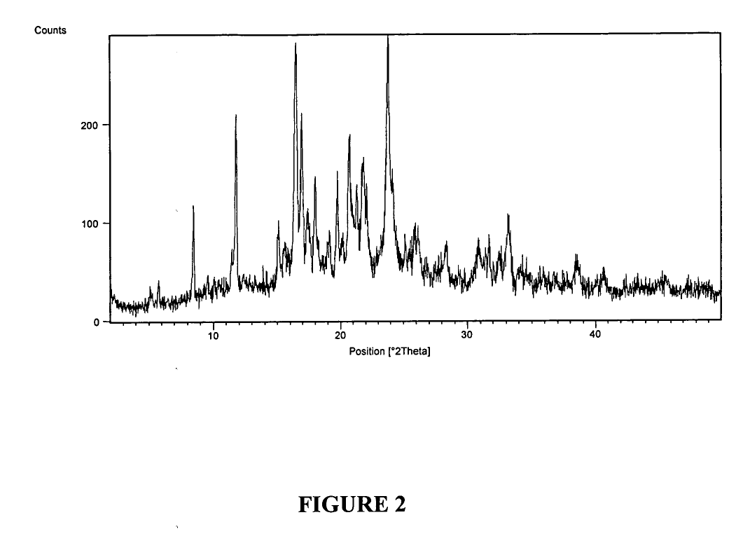

Another embodiment of the present invention is directed to crystalline Form V of desloratadine. The crystalline Form V of desloratadine has at least an XRD pattern substantially in accordance with FIG. 2. In one embodiment, desloratadine in polymorph Form V exhibits characteristic peaks (expressed in degrees 2θ±0.2°θ) at approximately one or more of the positions: about 15.07 and about 23.84. In another embodiment, desloratadine in polymorph Form V exhibits characteristic peaks (expressed in degrees 2θ±0.2°θ) at approximately one or more of the positions: about 8.46, about 11.80, about 15.07, about 16.95, about 18.00, about 19.75, about 20.66, about 21.86 and about 23.84. Generally, desloratadine in polymorph Form V exhibits an XRD pattern having the approximate values set forth in tabular form in Table 2:

TABLE 2

| | d-Value | Relative |
|---|---|---|
| 2θ | Angstrom | Intensity (%) |
| 8.46 | 10.45 | 41.85 |
| 11.80 | 7.50 | 64.26 |
| 15.07 | 5.35 | 82.54 |
| 16.95 | 5.23 | 67.87 |
| 17.40 | 5.10 | 26.97 |
| 18.00 | 4.93 | 43.76 |
| 19.08 | 4.65 | 14.95 |
| 19.75 | 4.50 | 42.33 |
| 20.66 | 4.30 | 55.42 |
| 21.86 | 4.07 | 49.01 |
| 23.84 | 3.73 | 100.00 |
| 26.04 | 3.42 | 15.80 |
| 28.36 | 3.15 | 15.41 |
| 30.86 | 2.90 | 16.59 |
| 33.20 | 2.70 | 26.81 |
| 38.53 | 2.34 | 10.93 |

Generally, crystalline Form V of desloratadine can be obtained by at least:

(a) heating a reaction mixture comprising loratadine and an inorganic base in an organic solvent to obtain a reaction mixture comprising desloratadine;

(b) heating the reaction mixture;

(c) extracting desloratadine in a water immiscible solvent;

(d) stirring the product of step (c) for about 2 to about 3 hours;

(e) optionally washing with an organic solvent and

(f) recovering desloratadine in crystalline Form V.

The inorganic base may be selected, but not limited to, alkali metal hydroxides such as lithium hydroxide, magnesium hydroxide, sodium hydroxide, potassium hydroxide, and the like. Useful organic solvents include, but are not limited to, alcohols, ketones, aromatic and aliphatic hydrocarbons, esters, ethers, nitrites, acids, water or mixtures thereof. The dissolution can be carried out at a temperature ranging from about 0° C. to about 150° C. and preferably at room temperature.

Useful water immiscible solvents include, but are not limited to, straight or branched chain aliphatic hydrocarbons, chlorinated solvents, cyclic solvents having 6 to 15 carbon atoms and the like and mixtures thereof. Exemplary water-immiscible solvents include, but are not limited to, dichloromethane, cyclohexane, n-heptane, hexane, octanol, n-decane, decalene and mixtures thereof. Isolation of the product can be achieved by techniques know in the art. For example, isolation may be accomplished by concentration by distillation, distillation under vacuum, or distillation in Rota evaporator, cooling, addition of a second solvent, filtration, filtration under vacuum, decantation and centrifugation or a combination thereof.

The product obtained may be further dried to achieve the desired residual solvent level. For example, the product may be further dried in a tray drier, or dried under vacuum and/or in a Fluid Bed Drier.

Yet another aspect of the present invention is directed to pharmaceutical compositions containing at least one or more of polymorph Forms III and V of desloratadine. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The polymorph Forms III and/or V of desloratadine of the present invention may also be administered as suppositories, ophthalmic ointments and suspensions, and parenteral suspensions, which are administered by other routes. The dosage forms may contain the polymorph Forms III and/or V of desloratadine of the present invention as is or, alternatively, may contain polymorph Forms III and/or V of desloratadine of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain the polymorph Forms III and/or V of desloratadine of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g., lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

In one embodiment, polymorph Forms III and/or V of desloratadine for use in the pharmaceutical compositions of the present invention can have a D₅₀ and D₉₀ particle size of less than about 400 microns, preferably less than about 200 microns, more preferably less than about 150 microns, still more preferably less than about 50 microns and most preferably less than about 15 microns. It is noted the notation Dₓ means that X % of the particles have a diameter less than a specified diameter D. Thus, a D₅₀ of about 400 microns means that 50% of the micronized particles in a composition have a diameter less than about 400 microns. The particle sizes of polymorph Forms III and/or V of desloratadine can be obtained by any milling, grinding micronizing or other particle size reduction method known in the art to bring the solid state polymorph Forms III and/or V of desloratadine into any of the foregoing desired particle size range.

Actual dosage levels of polymorph Forms III and/or V of desloratadine of the present invention may be varied to obtain an amount of the polymorph Forms III and/or V of desloratadine of the present invention that is effective to obtain a desired therapeutic response for a particular composition and method of administration. The selected dosage level therefore depends upon such factors as, for example, the desired therapeutic effect, the route of administration, the desired duration of treatment, and other factors. The total daily dose of the compounds of this invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc. The pharmaceutical compositions herein can formulated in any release form, e.g., immediate release, sustained release, controlled release, etc. A pharmaceutical composition of the invention will generally include about 0.1% by weight to about 99% by weight of the active ingredient, preferably about 1% to 50% by weight, based on the total weight of the composition.

The following examples are provided to enable one skilled in the art to practice the invention and are merely illustrative of the invention. The examples should not be read as limiting the scope of the invention as defined in the features and advantages.

### EXAMPLE 1

Preparation of Polymorph Form III of Desloratadine

A mixture of loratadine (10 g) and sodium hydroxide (11 g) in water (10 ml) and methanol (40 ml) was refluxed for about 6 to about 8 hours at a temperature ranging from about 78° C. to about 82° C. After completion of reaction as determined by TLC, the methanol was distilled out at a temperature of about 50° C., and then cooled to a temperature ranging from about 25° C. to about 30° C. until a thick slurry was obtained. Water (30 ml) and dichloromethane (40 ml) were added. The mixture was stirred at a temperature ranging from about 25° C. to about 30° C. for about 5 to about 10 minutes and the layers were allowed to settle. The organic layer was separated and the aqueous layer was further extracted with 2 portions of dichloromethane (50 ml). The combined organic layers were washed with water until a pH of about 7 to about 9 was achieved. The combined layers were then dried over sodium sulfate and stirred at a temperature ranging from about 25 to about 30° C. for about 96 hours. The solid separated was filtered, washed with chilled (0° C.) dichloromethane (2-5 ml) and dried at a temperature ranging from about 50° C. to about 60° C. under vacuum until loss on drying (“LOD”) was less than about 0.5% to give desloratadine Form III (about 4.8 g; yield—about 48% w/w).

The XRD of the final product is set forth in FIG. 1 and was recorded and identified as crystalline Form III of desloratadine.

### EXAMPLE 2

Preparation of Polymorph Form V of Desloratadine

A mixture of loratadine (10 g) and sodium hydroxide (11 g) in water (10 ml) and methanol (40 ml) was refluxed for about 6 to about 8 hours at a temperature ranging from about 78 to about 82° C. After completion of reaction as determined by TLC, water (40 ml) and dichloromethane (40 ml) were added. The mixture was stirred at temperature ranging from about 25° C. to about 30° C. for about 5 to about 10 minutes and the layers were allowed to settle. The organic layer was separated and the aqueous layer was further extracted with 2-3 portions of dichloromethane 50 ml. The combined organic layers were washed with water until a pH of about 8 was achieved. The solvent was distilled out until about 20 ml volume remained. The remaining mixture was stirred at a temperature ranging from about 15° C. to about 25° C. for about 2 to about 3 hours. The solid separated was filtered, washed with n-hexane (10 ml) and dried at a temperature ranging from about 50° C. to about 60° C. under vacuum until LOD was less than about 0.5% to give desloratadine Form V (about 3.0 g; yield—30% w/w).

The XRD of the final product is set forth in FIG. 2 and was recorded and identified as crystalline Form V of desloratadine

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. Desloratadine in polymorph Form III.

2. The desloratadine in polymorph Form III of claim 1, further characterized by having an X-ray powder diffraction (XRD) pattern substantially in accordance with FIG. 1.

3. The desloratadine in polymorph Form III of claim 1, further characterized by an XRD pattern having a characteristic peak (expressed in degrees 2θ±0.2°θ) at about 18.40.

4. The desloratadine in polymorph Form III of claim 1, further characterized by an XRD pattern having characteristic peaks (expressed in degrees 2θ±0.2°θ) at approximately one or more of the positions: about 7.10, about 7.74, about 8.20, about 10.67, about 11.47, about 17.08, about 17.97, about 18.40, and about 21.49.

5. A pharmaceutical composition comprising a therapeutically effective amount of desloratadine in polymorph Form III as defined in claim 1 and a pharmaceutically acceptable excipient.

6. A pharmaceutical composition comprising a therapeutically effective amount of desloratadine in polymorph Form III as defined in claim 2 and a pharmaceutically acceptable excipient.

7. A pharmaceutical composition comprising a therapeutically effective amount of desloratadine in polymorph Form III as defined in claim 3 and a pharmaceutically acceptable excipient.

8. The pharmaceutical composition of claim 5, in a solid dosage form.

9. The pharmaceutical composition of claim 5, in a form of a tablet, a caplet, a capsule, a suspension tablet, a troche, or a powder.

10. The pharmaceutical composition of claim 5, wherein the desloratadine in polymorph Form III has a D₅₀ and D₉₀ particle size of less than about 400 microns.

11. A process for preparing desloratadine in polymorph Form III, the process comprising:
(a) heating a reaction mixture comprising desloratadine;
(b) extracting desloratadine in a water immiscible solvent;
(c) stirring the product of step (b) for about 80 to about 100 hours; and
(d) recovering desloratadine in polymorph Form III.

12. The process of claim 11, wherein the desloratadine in polymorph Form III is further characterized by having an X-ray powder diffraction (XRD) pattern substantially in accordance with FIG. 1.

13. The process of claim 11, wherein the desloratadine in polymorph Form III is further characterized by an XRD pattern having a characteristic peak (expressed in degrees 2θ±0.2°θ) at about 18.40.

14. Desloratadine substantially in polymorph Form V.

15. The desloratadine in polymorph Form V of claim 14, further characterized by having an XRD pattern substantially in accordance with FIG. 2.

16. The desloratadine in polymorph Form V of claim 14, further characterized by an XRD pattern having characteristic peaks (expressed in degrees 2θ±0.2°θ) at about 15.07.

17. The desloratadine in polymorph Form V of claim 14, further characterized by an XRD pattern having characteristic peaks (expressed in degrees 2θ±0.2°θ) at about 8.46, about 11.80, about 15.07, about 16.95, about 20.66 and about 21.86.

18. A pharmaceutical composition comprising a therapeutically effective amount of desloratadine in polymorph Form V as defined in claim 14 and a pharmaceutically acceptable excipient.

19. A pharmaceutical composition comprising a therapeutically effective amount of desloratadine in polymorph Form V as defined in claim 15 and a pharmaceutically acceptable excipient.

20. A pharmaceutical composition comprising a therapeutically effective amount of desloratadine in polymorph Form V as defined in claim 16 and a pharmaceutically acceptable excipient.

21. The pharmaceutical composition of claim 18, in a solid dosage form.

22. The pharmaceutical composition of claim 18, in a form of a tablet, a caplet, a capsule, a suspension tablet, a troche, or a powder.

23. The pharmaceutical composition of claim 18, wherein the desloratadine in polymorph Form V has a D₅₀ and D₉₀ particle size of less than about 400 microns.

24. A process for preparing desloratadine in polymorph Form V, the process comprising:
(a) heating a reaction mixture comprising desloratadine;
(b) extracting desloratadine in a water immiscible solvent;
(c) stirring the product of step (b) for about 2 to about 3 hours; and
(d) recovering desloratadine in polymorph Form V.

25. The process of claim 24, wherein the desloratadine in polymorph Form V is further characterized by having an X-ray powder diffraction (XRD) pattern substantially in accordance with FIG. 2.

26. The process of claim 24, wherein the desloratadine in polymorph Form V is further characterized by an XRD pattern having characteristic peaks (expressed in degrees 2θ±0.2°θ) at about 15.07.
